(19) United States
(12) Patent Application Publication
(10) Pub. No.: US 2008/0248090 A1
(43) Pub. Date: Oct. 9, 2008

(54) GRADUATED CONCENTRATION SODIUM CHLORIDE PATCHES FOR THE TREATMENT OF DENTAL CONDITIONS

(76) Inventor: Gerard V. Sunnen, New York, NY (US)

Correspondence Address: OSTROLENK FABER GERB & SOFFEN, 1180 AVENUE OF THE AMERICAS, NEW YORK, NY, 100368403, US

(21) Appl. No.: 12/137,731

(22) Filed: Jun. 12, 2008

Related U.S. Application Data
(63) Continuation-in-part of application No. 11/197,239, filed on Aug. 3, 2005, now abandoned.
(60) Provisional application No. 60/678,892, filed on May 9, 2005.

(51) Int. Cl. A61K 9/70; A61K 33/14

(52) U.S. Cl. 424/447; 424/680

(57) ABSTRACT

A plurality of patches are prepared, containing a therapeutic agent, particularly NaCl, another salt, or another type of agent, for being held in contact with a portion of the oral cavity in order to treat a dental condition. The patches have respective concentrations of NaCl such that the concentrations cover a range from substantially hypotonic to substantially hypertonic. In particular, the plurality of patches may have a corresponding plurality of respective concentrations between about 0.22% and about 25% (weight/volume).

### CROSS-REFERENCE TO RELATED APPLICATIONS

This is a continuation-in-part of U.S. Ser. No. 11/197,239 filed Aug. 3, 2005, and entitled SODIUM CHLORIDE PATCH FOR TREATMENT OF DENTAL CONDITIONS, which claims the benefit and priority of U.S. Provisional Application No. 60/678,892 filed May 9, 2005, and entitled SODIUM CHLORIDE PATCH FOR THE PRIMARY, OR THE ADJUNCTIVE TREATMENT OF PERIODONTAL DISEASE, GINGIVAL INFECTIONS, AND OTHER DENTAL CONDITIONS, the disclosure of which is incorporated herein by reference.

### BACKGROUND

### Technical Field

This present disclosure relates to medication delivery systems, and more particularly to a system and a method for the efficient therapeutic application of sodium chloride or another therapeutic agent to dental tissues.

Periodontal disease affects millions of Americans and is a major public health concern worldwide. It is estimated that 15% to 20% of the adult population in the United States is afflicted with periodontal disease. The hallmarks of this condition are inflammation, infection, and bone regression. In advanced stages this may result in tooth loss.

Periodontal disease starts with gingivitis produced by bacterial plaque buildup along the gum line. Plaque, a mixture of food, saliva and bacteria, relentlessly grows if left unchecked and produces clefts or open pockets surrounding teeth. Toxins produced by bacteria weaken the ligaments binding teeth to gums.

Putative bacteria, or perio-pathogens, implicated in periodontal disease are mainly anaerobes and facultative anaerobes. Porphyromanas, Prevotela, Bacteriodes, Actinobacillus, fusiforms and spirochetes, Streptococcus, and Veillonella are regularly found in periodontal lesions.

Periodontal concretions are sources of microbial organisms that are rarely found elsewhere in the body. Capnocytophaga and Rothia, for example, once released in the circulation, are capable of producing bacteremias and infecting various organ systems as in infective endocarditis. It is therefore important to hold periodontal disease in check. Especially important is also the avoidance of bacteremias following dental procedures.

The therapy for periodontal disease consists of aggressive oral hygiene including scaling. Scaling is a procedure involving the scraping of plaque from the tooth. At times, scaling requires the penetration of dental instruments deeply below the gum line, resulting in irritation and bleeding. With the resultant disruption in tissue integrity and the presence of pathogens, bacterial invasion of the blood stream may ensue with possible ominous seeding into various organ systems.

Antibiotics are prescribed for dental infections including abscess formation. Serial antibiotics, however, may pose disadvantages to overall health, with the possible occurrence of gastro-intestinal dysfunction, fatigue, and the weakening of immune resilience. Gargles are prescribed to inhibit infection, to reduce the swelling of gums, and to relieve pain. Gargles reduce the need for serial antibiotic prescriptions.

Sodium chloride is a universal bacteristatic, bacteriocidal, anti-viral, and anti-fungal agent. It possesses anti-inflammatory properties and has a well-known safety profile when applied topically. All the above bacterial species are inhibited and inactivated by sodium chloride.

Sodium chloride gargles are often prescribed for purposes of enhancing oral hygiene. Importantly, they are indicated as adjunctive therapy in the management of periodontal disease, dental abscesses, and post dental surgery including endodontic procedures. Gargles, however, exert limited effectiveness because of the relatively low tolerance patients have for them.

While the strategy of applying therapeutic solutions to control periodontal and oral infections—as in gargles—has solid value, its effectiveness is only marginally exploited.

The reason for this is that gargle solutions have only a limited time of patient tolerability. Patients can keep solutions in their oral cavities for limited time frames, usually counted in terms of seconds, before they feel the need to expel them. Within that time, the solutions cannot adequately perfuse the dental pockets and penetrate deeply into the gum tissues to exert their therapeutic effects.

In the case of sodium chloride gargles, for example, gargling will rapidly stimulate and then irritate receptors within the throat that will induce expulsive reflexes.

Sodium chloride solutions, in addition, require sufficient time of exposure to perform their anti-inflammatory, bacteriostatic, bactericidal, and anti-fungal actions. Gargling alone cannot achieve this task since the required time for effective sodium chloride exposure to effectively diffuse through layers of dental tissues ranges from several minutes to at least half an hour or more.

### SUMMARY

To avoid the foregoing disadvantages of known NaCl delivery systems, disclosed herein is a system of sodium chloride applicator patches and a method of using the same for the primary or the adjunctive treatment of periodontal disease, gingival infections, and other dental conditions. The patches make possible the prolonged application of sodium chloride to oral tissues. The patches are impregnated with sodium chloride, in either its solid or liquid form. Several options for this impregnation are described. Other therapeutic agents may also be included. The patches may maintain their long-term integrity by enclosure in a hermetic package.

When the patch is applied to dental tissues, the sodium chloride diffuses within the tissues to exert its therapeutic functions.

Several concentrations of sodium chloride are available, offering a spectrum of therapeutic options. Other salts, including magnesium chloride, potassium chloride, zinc chloride, and calcium chloride, among others, may be added to the sodium chloride to reach optimal therapeutic effects.

The patch, which is configured in different sizes—including configurations that are capable of treating an entire dentition—is directly applied to the gum tissues under treatment. According to one embodiment, the patch may have incorporated micro suction cups to hold it in position.

As best understood, the mechanism of the invention's therapeutic action is as follows.

One of the proven and time-tested remedies for dental irritation, inflammation, and infection is the application of sodium chloride solution of various degrees of saturation. Indeed, bacteria and fungi cannot maintain their intracellular osmotic balance in the presence of salt solutions. Cellular functions cease, cellular membranes disintegrate, and pathogen death occurs.

The mechanisms of sodium chloride's anti-viral action are less clear. Viruses, however, like most organisms, need a hospitable milieu in which to thrive.

Sustained exposure to sodium chloride solution allows for deep penetration of a hyper-osmotic extra-cellular milieu to progressively perfuse ever further into gum tissues, eventually reaching the deepest gum pockets, inactivating their bacterial concretions.

The dental patch may be positioned on any gum or tissue within the oral cavity, possibly by means of incorporated micro-suction cups, adhesives or other suitable means. The patch may be placed on the medial or on the lateral side of dental structures, or on both, and may extend far up into the palate. Importantly, its effects may thus reach into the peri-root regions of the teeth. Indeed, the root end opening is consistently prone to irritative and infective processes often produced by problems associated with root canal therapy, including apicoectomy.

Once the patch is put in place, the sodium chloride diffuses from its matrix into the tissues under treatment. The rate of diffusion is dependent upon the relationship between the matrix and its sodium chloride content.

According to various embodiments, an applicator patch and a method are provided for applying salt solutions, allowing for the prolonged therapeutic exposure of the solutions to dental (gingival and teeth), and other tissues.

Preferred aspects of the patch may comprise the following:
A. A matrix capable of holding sodium chloride, alone or in combination with other salts, in their solid, gel, or liquid forms. Other salts include, but are not limited to, magnesium chloride, potassium chloride, zinc chloride, and calcium chloride.
B. The matrix may be constructed of rubber-based products, silicone, polymer compounds, ceramic compounds, or fabrics, for example.
C. The matrix may hold the salt via chemical bonds.
D. The matrix may hold the salt solution through permeation of the matrix.
E. The matrix gradually releases its salt content on contact with tissues.
F. The matrix may be dotted with micro-suction cups, allowing it to reversibly stick to dental tissues, including teeth, and other tissues. These micro-suction cups may be constructed of silicone, or other materials capable of forming a hermetic suction seal with the skin.
G. The patch may be lined, on its posterior surface, with an impermeable surface.
H. The patch may be disposable.

Preferred aspects of the method may comprise the following:
1. The application of salt solutions to dental tissues for prolonged periods of time (spanning from a few minutes to several hours) as a primary therapy, or as an adjunctive therapy for a spectrum of conditions which may include, but are not limited to: periodontal disease; gingival infections including abscesses; the management of the aftermath of dental procedures such as scaling, extractions, gum surgery, and endodontic surgery including apicoectomy.
2. While the method in this invention emphasizes the use of salt solutions, and in particular sodium chloride, the patch and method may have a variety of uses such as, but not limited to: the administration of dental anesthesia; the dental application of bacteristatic and bacteriocidal solutions other than sodium chloride; the application of anti-pyretic and anti-inflammatory solutions; and the application of tooth-strengthening solutions such as fluoride.
3. A method of applying a graduated series of sodium chloride concentrations to dental tissues, predicated upon patient requirements. Envisioned, for example, is a spectrum of sodium chloride patches of concentrations ranging from a physiological concentration (0.9%), up to perhaps 40% saturated solutions, offered in 5% increments, for example.

More specifically, a series of patches have graduated concentrations of sodium chloride that range from hypotonic (say 0.5%) to increasing gradations of hypertonicity. This system allows the health care provider a patient-specific prescription palette for treating a variety of dental disorders.

“Normal saline” reflects a concentration of sodium chloride corresponding to those found in bodily tissues. Normal saline measures 0.9% sodium chloride (weight/volume). Human blood contains 0.9 grams of sodium chloride per 100 milliliters of serum.

“Hypertonic” saline signifies any sodium chloride solution of higher concentration than normal saline. Hypertonic saline solutions range from 0.9% to as high as 25% or more. At elevated concentrations, the solutions have a tendency to deteriorate and may need stabilizers to maintain their integrity. Said stabilizers, which aim to enhance the ionic balance and/or polarity of the solution, may consist of other salts (e.g., potassium chloride; magnesium chloride; calcium carbonate), and/or organic molecules (e.g., dextran).

The system of graduated concentration sodium chloride patches allows for proper dosing relative to the condition under treatment. For example, a spectrum of saline concentrations may be provided, ranging from hypotonic quarter normal saline (0.22%), half normal saline (0.45%) (0.5% saline), to isotonic saline (0.9%), thereafter increasing in intervals of 1%, until reaching a maximum of 25%.

The health care provider, with this system, can take advantage of a wide range of prescription options. Some patients, for example, who have undergone dental surgery or who suffer from acute infections may initially be prescribed higher saline concentrations in order to counter tissue swelling, or to counter bacterial growth, then move on, in serial fashion, to lower concentration levels as tissues heal. In periodontal disease, low concentrations may initially be chosen to allow the patient to adapt to the therapy, gradually inching to higher levels as adjustment progresses.

The benefits of applying hypertonic saline solutions to tissues such as gums, mucous membranes, other buccal tissues, and tissues in general are many. Among them are:
a) A reduction of the swelling of inflamed tissues. Salt draws water. Inflamed tissues, exposed to salt, lose their engorgement with fluids and are coaxed to return to their normal architecture.
b) Hypertonic sodium chloride solutions are antibacterial, antifungal, and antiviral. Bacteria and fungi exposed to hypertonic solutions are faced with an inhospitable growth medium. Due to the disruption of their osmotic equilibrium, they cease to function and reproduce. Bacterial and fungal death ensues. Mechanisms of viral death are less clear. In another phenomenon, called “bacterial communication,” the salty milieu challenges the bacterial colony, leading to its global inhibition via signals bacteria send to each other. In this model, a salt challenge to one part of the bacterial colony will be transmitted to the entire colony, leading to the regression of the infection process.
c) Hypertonic salt solutions, through their action of reducing the turgor of swelled tissues, allow for the increased circulation of said tissues. Restoring circulation is essential for the delivery of oxygen and immune infection-fighting components, and for the removal of infection-derived toxins.
d) Hypertonic salt solutions prevent leucocytes from adhering to tissues, thus immunologically inhibiting excess inflammation. They also counteract the action of excitatory amino acids that promote inflammation.

Other features and advantages will become apparent from the following description of embodiments thereof, which refers to the accompanying drawings.

### BRIEF DESCRIPTION OF THE DRAWINGS

FIG. 1 is a plan view of a dental patch.

FIG. 2 is a lateral view of the dental patch.

FIG. 3 shows a hermetic sachet for enclosing the patch, in plan view.

FIG. 4 is a ¾ view of an open hermetic sachet as seen from its open end.

FIG. 5 shows an application of the patch to teeth and gums.

### DETAILED DESCRIPTION

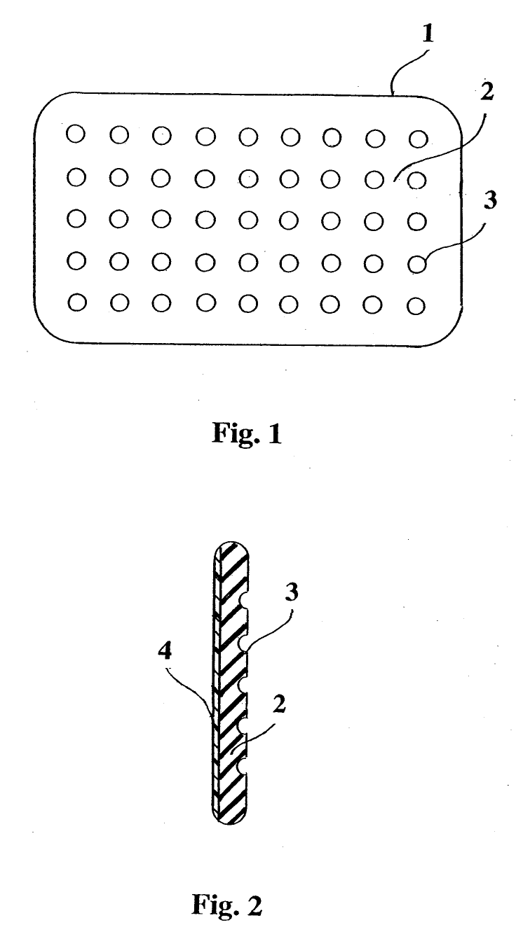

FIG. 1 is a plan view of a dental patch. Illustrated is a rectangular patch (measuring perhaps 2×3 centimeters) (1). Other sizes and configurations are also usable in response to therapeutic demands. The matrix (2) contains sodium chloride. Incorporated within the matrix of the patch is a series of micro-suction cups (3) which, when pressed against dental tissues, including teeth, allow the patch to stay in place.

FIG. 2 is a lateral view of the dental patch. The thickness of the patch may be about 0.5 centimeters. Patch thickness, however, may range from a millimeter to a centimeter or more, again in response to therapeutic requirements. A lateral view of the micro-suction cups (3) is shown, as well as their relation to the matrix (2). On the back of the patch is a sheet of material (4) impervious to the contents of the matrix.

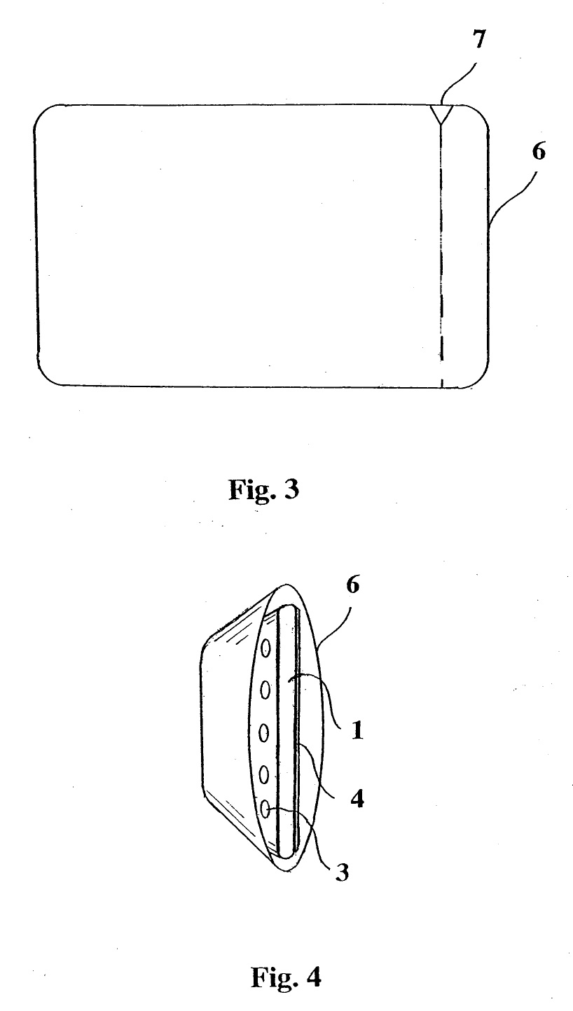

FIG. 3 shows a hermetic sachet (6) for enclosing the patch, in plan view. A nick (7) in the sachet allows for convenient opening.

FIG. 4 is a ¾ view of an open hermetic sachet (6) as seen from its open end. The dental patch (1) is dotted with micro-suction cups (3) on one side, and a barrier impervious to liquids on the other (4).

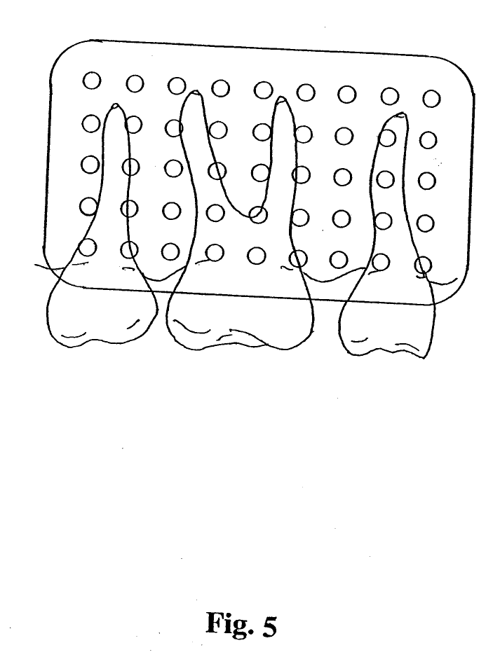

FIG. 5 shows the actual application of the dental patch to teeth and gums.

The dental patch is composed of a pliable matrix composed of materials capable of absorbing sodium chloride in its solid, gel, or liquid form.

The sodium chloride may exist in the form of a chemical bond with the chemical composition of the matrix. Such chemicals may include, but are not limited to, rubber-based compounds, plastic compounds, ceramic compounds, or fabrics.

Alternatively, the matrix holding the sodium chloride may do so through absorptive processes. The sodium chloride saturating the matrix may exist in its solid form, or in the form of solutions, or of gels. Absorptive materials may include fabrics, rubber-based compounds, ceramic compounds, or plastic compounds.

Therapeutic salt solutions may include, but are not limited to, sodium chloride. Other salts may be used alone, or in conjunction with sodium chloride including, but not limited to, potassium chloride, magnesium chloride, magnesium carbonate, zinc chloride, and calcium chloride.

The patch's sodium chloride concentrations are available in gradients of concentration. Specific gradients may be indicated for different clinical conditions. Sodium chloride concentrations may range from close to the physiological (0.9%), to peri-saturation levels in water (35%- to 40%). Incorporated within gels, the salt concentrations may be higher.

Suggested salt concentrations in patches start at 5% and are available in increments of 5%, up to 40%.

The sizes and configuration of the patches are adapted to dental needs.

The back of the patch is lined with a material impervious to fluids so that the sodium chloride application does not appose itself to tissues not intended for treatment.

The patch is disposable.

More specifically, a series of patches have graduated concentrations of sodium chloride that range from hypotonic (say 0.5%) to increasing gradations of hypertonicity. This system allows the health care provider a patient-specific prescription palette for treating a variety of dental disorders.

“Normal saline” reflects a concentration of sodium chloride corresponding to those found in bodily tissues. Normal saline measures 0.9% sodium chloride (weight/volume). Human blood contains 0.9 grams of sodium chloride per 100 milliliters of serum.

“Hypertonic” saline signifies any sodium chloride solution of higher concentration than normal saline. Hypertonic saline solutions range from 0.9% to as high as 25% or more. At elevated concentrations, the solutions have a tendency to deteriorate and may need stabilizers to maintain their integrity. Said stabilizers, which aim to enhance the ionic balance and/or polarity of the solution, may consist of other salts (e.g., potassium chloride; magnesium chloride; calcium carbonate), and/or organic molecules (e.g., dextran).

The system of graduated concentration sodium chloride patches allows for proper dosing relative to the condition under treatment. For example, a spectrum of saline concentrations may be provided, ranging from hypotonic quarter normal saline (0.22%), half normal saline (0.45%) (0.5% saline), to isotonic saline (0.9%), thereafter increasing in intervals of 1%, until reaching a maximum of 25%.

The health care provider, with this system, can take advantage of a wide range of prescription options. Some patients, for example, who have undergone dental surgery or who suffer from acute infections may initially be prescribed higher saline concentrations in order to counter tissue swelling, or to counter bacterial growth, then move on, in serial fashion, to lower concentration levels as tissues heal. In periodontal disease, low concentrations may initially be chosen to allow the patient to adapt to the therapy, gradually inching to higher levels as adjustment progresses.

The benefits of applying hypertonic saline solutions to tissues such as gums, mucous membranes, other buccal tissues, and tissues in general are many. Among them are:
a) A reduction of the swelling of inflamed tissues. Salt draws water. Inflamed tissues, exposed to salt, lose their engorgement with fluids and are coaxed to return to their normal architecture.
b) Hypertonic sodium chloride solutions are antibacterial, antifungal, and antiviral. Bacteria and fungi exposed to hypertonic solutions are faced with an inhospitable growth medium. Due to the disruption of their osmotic equilibrium, they cease to function and reproduce. Bacterial and fungal death ensues. Mechanisms of viral death are less clear. In another phenomenon, called “bacterial communication,” the salty milieu challenges the bacterial colony, leading to its global inhibition via signals bacteria send to each other. In this model, a salt challenge to one part of the bacterial colony will be transmitted to the entire colony, leading to the regression of the infection process.
c) Hypertonic salt solutions, through their action of reducing the turgor of swelled tissues, allow for the increased circulation of said tissues. Restoring circulation is essential for the delivery of oxygen and immune infection-fighting components, and for the removal of infection-derived toxins.
d) Hypertonic salt solutions prevent leucocytes from adhering to tissues, thus immunologically inhibiting excess inflammation. They also counteract the action of excitatory amino acids that promote inflammation.

Although particular embodiments have been described, many other variations and modifications and other uses will become apparent to those skilled in the art. Therefore, the present invention is not limited by the specific disclosure herein.

What is claimed is:

1. A system comprising a plurality of patches for the therapeutic application of NaCl to dental tissue, each of said patches comprising:
a matrix capable of holding NaCl, and NaCl held in the matrix; and
a holding structure on the matrix capable of holding the matrix in contact with dental tissue;
said patches having mutually distinct respective concentrations of NaCl.

2. The system of claim 1, wherein said concentrations cover a range from substantially hypotonic to substantially hypertonic.

3. The system of claim 1, wherein said plurality of patches have a corresponding plurality of respective concentrations between about 0.22% and about 25% (weight/volume).

4. The system of claim 1, wherein said NaCl is held in said matrix in solid, gel or liquid form.

5. The system of claim 1, further comprising at least one additional salt held in said matrix, selected from the group consisting of potassium chloride, magnesium chloride, magnesium carbonate, zinc chloride, calcium chloride and mixtures thereof.

6. The system of claim 1, wherein said matrix comprises a material selected from the group consisting of rubber-based products, silicone, polymer compounds, ceramic compounds, and fabrics.

7. The system of claim 1, wherein the NaCl is held in the matrix by a chemical bond.

8. The system of claim 1, wherein the NaCl is absorbed into the matrix.

9. The system of claim 1, further comprising a therapeutic agent held in the matrix; wherein said therapeutic agent is selected from the group consisting of dental anesthesia; bacteristatic and bacteriocidal solutions other than sodium chloride; anti-pyretic and anti-inflammatory solutions; and tooth-strengthening solutions such as fluoride.

10. The system of claim 9, wherein said agent is incorporated in said matrix in a concentration of about 0.9% to about 40%.

11. A method utilizing a plurality of patches for the application of NaCl to dental tissue, said application being performed by the steps of:
preparing a plurality of patches incorporating the NaCl in a matrix; and
adhering one of said patches to the dental tissue to be treated;
said one patch being selected from among a prepared plurality of patches having respective mutually distinct concentrations of NaCl.

12. The method of claim 11, wherein said concentrations cover a range from substantially hypotonic to substantially hypertonic.

13. The method of claim 12, wherein said plurality of patches have a corresponding plurality of respective concentrations between about 0.22% and about 25% (weight/volume).

14. The method of claim 11, wherein said NaCl is held in said matrix in solid, gel or liquid form.

15. The method of claim 11, wherein said matrix further contains at least one salt held in said matrix, selected from the group consisting of potassium chloride, magnesium chloride, magnesium carbonate, zinc chloride, calcium chloride and mixtures thereof.

16. The method of claim 11, wherein said matrix further contains a therapeutic agent selected from the group consisting of dental anesthesia; bacteristatic and bacteriocidal solutions other than sodium chloride; anti-pyretic and anti-inflammatory solutions; and tooth-strengthening solutions such as fluoride.

17. The method of claim 16, wherein therapeutic agent is contained in a concentration of about 0.9% to about 40%.
